(19) United States
(12) Patent Application Publication
(10) Pub. No.: US 2020/0132683 A1
(43) Pub. Date: Apr. 30, 2020

(54) RAPID MULTIPLEX DIAGNOSTIC FOR PARKINSON'S DISEASE AND ALZHEIMER'S DISEASE

(71) Applicant: Integrated Neurologics LLC, Detroit, MI (US)

(72) Inventors: Mark Wasserman, Detroit, MI (US); Todd Flower, W. Bloomfield, MI (US)

(21) Appl. No.: 16/664,341

(22) Filed: Oct. 25, 2019

Related U.S. Application Data
(60) Provisional application No. 62/750,688, filed on Oct. 25, 2018.

(51) Int. Cl. G01N 33/543; G01N 31/22

(52) U.S. Cl. CPC G01N 33/54346; G01N 2800/2821; G01N 2800/2835; G01N 31/22

(57) ABSTRACT

A method for diagnosing Parkinson's and Alzheimer's disease without invasive techniques or complicated wet-laboratory equipment. The method utilizes the rapid detection of both the CD38 protein and alpha-synuclein protein at a specific concentration that reflects the pathological state of Parkinson's disease, and CD38 protein and the tau protein which are found in significantly elevated concentrations in blood plasma and serum in the disease states. The blood plasma or serum can be collected and utilized as the test material for the pre- and post-symptom Parkinson's disease and Alzheimer's disease.

### CROSS-REFERENCE TO RELATED APPLICATIONS

This application claims the benefit of U.S. Provisional Application No. 62/750,688, filed Oct. 25, 2018. The subject matter all of which is incorporated herein by reference.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The present invention relates to the field of medicine and wellness, and more specifically, to diagnosis for neurodegenerative disorders, particularly Parkinson's disease and Alzheimer's disease.

### BACKGROUND

Parkinson's disease is the second most common neurodegenerative disease following Alzheimer's disease, affecting approximately 1% of people over 65 years and 5% of those over 85. Parkinson's disease is a progressive neurodegenerative movement disorder resulting from the loss of dopaminergic neurons in the midbrain. The main pathological hallmarks of Parkinson's disease affect motor functions, including resting tremor, rigidity and loss of postural reflexes. Additionally, non-motor signs, such as depression, sleep disorders, dementia and peripheral impairments are recognized to precede and/or occur with the progressive loss of the dopaminergic neurons. This loss is coupled with the accumulation of protein aggregates of alpha-synuclein into intraneuronal structures (called Lewy bodies and Lewy neurites). The first motor disturbances are not observed until the loss of dopaminergic neurons in the Substantia Nigra pars compacta reaches almost 70% and at least 80% loss of dopamine in the striatum. The preclinical phase of the progressive dopaminergic neuron degeneration before the onset of symptoms is estimated to last 8-17 years, implicating the existence of compensatory mechanisms in early Parkinson's disease.

Therefore, the search for preclinical PD biomarkers represents a crucial goal to design future neuroprotective therapies for at-risk populations, aimed at delaying and/or limiting the ongoing degeneration process before the appearance of the first clinical symptoms. Additionally, this might lead to the identification of novel molecular targets for the development of more effective drugs for this disease. The causes and mechanisms contributing to dopaminergic neuron degeneration in Parkinson's disease are not well defined and, currently, there is no cure for Parkinson's disease but only treatments able to relieve the symptoms and to improve quality of life. These treatments include: supportive therapies (such as rehabilitation, via physiotherapy, occupational therapy and speech and language therapy) and palliative medications (e.g., the Dopamine precursor levodopa (L-DOPA), dopamine agonists, catechol-O-methyl transferase (COMT) inhibitors, monoamine oxidase B (MAO-B) inhibitors, amantadine and anti-cholinergic molecules and immunomodulatory therapies). If the palliative drugs fail to adequately control patients' symptoms, deep brain stimulation can be used. Deep brain stimulation utilizes a surgically implanted neurostimulator able to deliver electrical stimulation to targeted areas in the brain that control movement, blocking the abnormal nerve signals that cause tremor, rigidity and walking problems.

The majority of Parkinson's disease cases have been referred to as sporadic (idiopathic Parkinson's disease), thus underlying a critical interplay between genetic susceptibility and environmental factors. However, a mutation that identified the first Parkinson's disease-linked mutation, alpha-synuclein (A53T) in Italian and Greek origin families found that four persons with notable parkinsonism after intravenous injection of an illicit drug, containing 1-methyl-4-phenyl-1,2,5,6-tetrahydropyridine (MPTP) in 1983. Soon afterward, a series of missense point mutations in the N-terminal of alpha-synuclein, including A30P, E46K, H50Q, G51D and A53E, were also identified. Alpha-synuclein is the main component in Lewy bodies and is phosphorylated at Ser129 of alpha-synuclein, which facilitates α-synuclein fibril uptake by neurons and exacerbated the pathology progression of Parkinson's disease.

Additionally, it has been discovered that the dosage of the alpha-synuclein gene (SNCA) is responsible for parkinsonism. Point mutation and triplication carriers show almost complete penetrance, whereas penetrance in duplication carriers ranges between 30% and 50%. What's more, the alpha-synuclein gene (SNCA) triplication carriers also tend to have an even earlier onset and a more severe phenotype than the duplication carriers. This data is strong evidence that dysfunction and accumulation of α-Syn plays a key role in both familial and sporadic forms of PD. In addition to LB inclusions, α-Syn has been shown to accumulate in axons and in the presynaptic terminal suggesting these pathogenic accumulations are a key trigger of onset and formation of PD-related synaptopathy.

Alzheimer's disease (AD) is a genetically complex, multifactorial disease that leads to neurodegenerative dementia. Patients display a progressive decline of cognitive capabilities, with characteristic early loss of episodic memory, eventually resulting in complete dependency and death. The disease is preceded by a long prodromal phase. AD is associated with regional cerebral hypometabolism, extracellular Aβ plaques, intracellular neurofibrillary tangles (NFTs) containing hyperphosphorylated tau, neuroinflammation and oxidative stress, loss of synaptic connections, neural death and atrophy and resultant clinical manifestations of AD. There are four major pathological processes that cause the pathogenesis of Alzheimer's disease:

First, mitochondrial dysfunction is manifested primarily by reduction of energy metabolism which in turn negatively affects all cellular functions. An excessive production of reactive oxygen species (ROS) produces oxidative stress if compensating mechanisms are inadequate. The pathologic process producing mitochondrial dysfunction appears to be associated with aging, from maternally transmitted mitochondrial DNA mutations or spontaneous genetic mutations.

Secondly, abnormal protein deposits (inclusions) are damaging to neural elements. Familial AD is associated with hereditary mutations which affect critical regions of amyloid-β protein precursor (AβPP). Amyloid pathologies also increased in late onset AD where coalesced inclusions ultimately formed senile plaques. Additionally, neurofibrillary tangles, made up of pathologic tau proteins, are found in all later stages of the disease. A more recently identified inclusion, TAR DNA-binding protein 43 (TDP-43, transactive response DNA binding protein 43 kDa), seen in fronto-temporal degeneration, is also estimated to be present in 50% of Alzheimer's patients.

Thirdly, oxidation can be destructive to any structures whose components may be oxidized. Structure and component elements of cells, including membranes, organelles and DNA can be oxidized and lose their structural integrity and physiologic function. In this process of oxidative stress reactive oxygen species are not neutralized. This can occur when ROS are increased and overwhelm the defensive elements or when there is a decrease in the latter, as with aging. Advanced glycation end products and their receptors constitute a process seen in aging and is shown to be present in Alzheimer's disease to a much greater degree than in unaffected individuals.

Finally, neuroinflammation can occur in association with microglia or astrocytic dysfunction. These cells tend increasingly to convert from anti-inflammatory to pro-inflammatory activity with aging. Abnormalities may be created by the release of pro-inflammatory immune cytokines (e.g., interleukin-1 (IL-1), tumor necrosis factor alpha (TNF-α)), with immunological deficiencies promoting neurodegeneration.

Deposition of amyloid-β (Aβ) peptide in the brain is considered to represent the primary event in AD, and the amyloid precursor protein (APP), a trans-membrane receptor, is central to the pathogenesis though its exact function is unknown. Aβ is generated by sequential proteolytic cleavage of APP by β-secretase or B-site APP cleaving enzyme (BACE)-1 from within and by γ-secretase from outside the membrane. When normally soluble Aβ peptides attain a definite level, they become insoluble, misfold and aggregate into Aβ plaques. These plaques are composed of insoluble peptides, generally 42 amino acids in length (Aβ1-42) and the oligomeric forms of Aβ1-42 are thought to have a greater neurotoxic potential than monomers or fibrils. Cleavage of APP by the α-secretase followed by γ-secretase activates the neuroprotective amyloid precursor protein (APPα).

Alzheimer's disease is associated with higher membrane-associated free cholesterol and greater overall brain cholesterol load. Genetic mutations of amyloid precursor protein (chromosome 21) or trisomy 21 cause early onset autosomal dominant Alzheimer's disease. Mutations of presenilin-1, presenilin-2, clusterin, phosphatidylinositol-binding clathrin assembly protein, complement component (3b/4b) receptor 1 and triggering receptor expressed on myeloid cells 2 have also been found to be associated with the disease. While increased production of Aβ may cause early-onset Alzheimer's disease, late onset Alzheimer's disease may be caused by impaired Aβ clearance due to interactions with ApoEϵ4, reduced proteolysis, decreased transport across the blood-brain barrier or inefficient cerebrospinal fluid transport.

In particular, aging, inflammation and exposure to neurotoxic agents have all been identified as contributors to neurodegeneration, and the dopaminergic neuronal loss seen in Parkinson's disease. Several molecular pathways have been implicated in the pathogenesis of Parkinson's disease and Alzheimer's disease. These include oxidative stress, mitochondrial impairment, neuroinflammation and disruption of the ubiquitin-proteasome machinery. As ageing is a common denominator in many neurodegenerative conditions, factors that contribute to ageing may also contribute to the progression of neurodegenerative diseases. Of upmost importance to this relationship of cellular ageing and neurodegeneration is the importance of NAD+biosynthesis for the pathophysiologies of aging and aging-related diseases.

NAD+is an essential component of cellular processes necessary to support various metabolic functions. The classic role of NAD+is a co-enzyme that catalyzes cellular redox reactions, becoming reduced to NADH in many fundamental metabolic processes, such as glycolysis, fatty acid beta oxidation, or the tricarboxylic acid cycle. In addition to playing these roles, NAD+has a critical role as the substrate of NAD+-consuming enzymes such as poly-ADP-ribose polymerases (PARPs), Sirtuins and CD38/157 ectoenzymes.

It is now becoming a consensus that NAD+levels decline at cellular, tissue/organ, and organismal levels during aging. Activities of NAD+-consuming enzymes are affected by this NAD+decline, contributing to a broad range of age-associated pathophysiologies. There are several factors that can be attributed to the decrease in NAD+levels, but of particular interest for Parkinson's disease and Alzheimer's disease is the activity of the primary cellular NADase, CD38.

The activity of CD38 mainly generates ADPR and nicotinamide by hydrolysis of NAD+, it has a secondary role to mediate cellular signaling through the generation of cyclic ADPR (cADPR), a potent Ca+2 inducer. CD38 can also degrade the NAD+precursors, NMN and NR, as well as NAD+, thus modulating cellular NAD+content. Additionally, it has been reported that CD38 protein levels increase in multiple tissues and organs over age, contributing to NAD+decline. Therefore, CD38-dependent modulation of NAD+can alter the activity of NAD+-consuming enzymes and affect cellular signaling and metabolism.

The relationship between CD38, NAD+levels and Parkinson's disease and Alzheimer's disease can ultimately be tied to a family of NAD+-dependent enzymes known as sirtuins. Sirtuins are a family of nicotinamide adenine dinucleotide (NAD+)-dependent deacylases with remarkable abilities to prevent diseases and even reverse aspects of ageing. The sirtuins also serve many additional roles, including the control of energy metabolism, cell survival, DNA repair, tissue regeneration, inflammation, neuronal signaling and even circadian rhythms

Clear evidence exists that demonstrates that the sirtuins play an integral role in suppressing Parkinson's disease: SIRT1 has been demonstrated to protect cells against alpha-synuclein-induced toxicity by upregulation molecular chaperones. Additionally, resveratrol, a small molecule activator of SIRT1, ameliorated both motor deficits and pathological changes in MPTP-treated mice via activation of SIRT1 and subsequent LC3 deacetylation-mediated autophagic degradation of alpha-synuclein. SIRT2 is the most abundant sirtuin in the brain, and its levels increase with ageing. SIRT2 also appears to play a role in the pathogenesis of Parkinson's disease in that it modulates the aggregation and toxicity of alpha-Syn, but the molecular mechanism remains unclear. However, the balance between acetylation and deacetylation is altered in both ageing and neurodegeneration. It has also been demonstrated that SIRT2 interacts with and de-acetylates α-Syn at residues K6 and K10 leading to decreased aggregation and toxicity. Lastly, SIRT3, a mitochondrial sirtuin, plays a critical role in mitochondrial function. It has recently been demonstrated that SIRT3 overexpression in a PD cell culture model results in increased cell viability, a decrease in apoptosis, prevention of α-Syn aggregation and decreased ROS generation.

Individuals with Alzheimer's disease have decreased expression levels of SIRT1 which have been reproduced in the hippocampus of Alzheimer's disease model mice. SIRT1 activation is capable of reducing the amount of oligomerized amyloid beta through upregulating the production of alpha secretase. Additionally, SIRT1 promotes neuronal function and survival in AD model mice. CA1-localized SIRT1 overexpression not only preserves learning and memory in AD mice but enhances cognitive function in non-AD model mice. Another study demonstrated that CD38 regulates Alzheimer's disease pathology by using a CD38 null mouse model of Alzheimer's disease. This study specifically demonstrated that in a CD 38 null mouse, mice exhibited significant reductions in Aβ plaque load and soluble Aβ levels compared to APP.PS mice. These data imply a direct relationship between CD38, NAD+and the pathogenesis of Alzheimer's disease.

There is unequivocal evidence that alpha-synuclein plays a pivotal pathophysiological role in neurodegenerative diseases, and in particular in synucleinopathies. Alpha-synuclein, an aggregation-prone and amyloid-forming protein, plays a pivotal role in the pathogenesis of synucleinopathies such as Parkinson's disease, dementia with Lewy bodies and multiple system atrophy. Recently, the aggregation of disease-related proteins in physiological aging has been attributed to altered protein homeostasis (proteostasis).

Though alpha-synuclein is abundantly expressed in the human brain, neuronal release causes its presence in extracellular biological fluids, such as cerebrospinal fluid (CSF), plasma and serum as evidenced by previous studies. Since alpha-synuclein is one of the major constituents of cytoplasmic Lewy bodies, it is potentially a target biomarker for Parkinson's disease. Several studies have found significantly higher levels of alpha-synuclein in Parkinson's disease patients when compared with controls. In addition, the increase in alpha-synuclein plasma levels correlated with the progression of the disease.

The Tau protein primarily binds to microtubules and helps to stabilize them. It has been well established that the detachment of the tau proteins from the microtubules results in the formation of the neurofibrillary tangles which contribute to Alzheimer's disease dementia. Numerous potential biomarkers are currently under investigation for Alzheimer's disease, among which tau and beta amyloid have been considered since the concentration of tau in both the brain and Cerebrospinal fluid is much higher in Alzheimer's compared to controls. It has also been reported that these same concentration differences have been observed with respect to tau protein levels in the blood. In fact, it has been established that that tau protein levels in control groups are on average 6 times less than that of Alzheimer's disease subjects (control 9.99 ng/ml, Alzheimer's disease 61.91 ng/ml). This is well within the detectable range of lateral flow.

Additionally, Alzheimer's disease is pathologically characterized by the massive deposition of abnormal protein aggregates present as extracellular plaques of amyloid-β peptide (Aβ) in the brain parenchyma. It is widely believed that abnormal levels of Aβ trigger the disease [96]. Aβ is a small hydrophobic 4 kDa peptide, which is derived by sequential proteolytic processing of the amyloid precursor protein (APP), a single transmembrane protein with type I topology which is expressed in neurons as a 695 amino acid splice variant. The cleavage product is then deposited into plaques. These plaques are neurotoxic aggregates believed to be the culprit of AD pathogenesis.

Therefore, a need exists to improve over the prior art and more particularly, for a rapid cost-effective diagnostic indicator for Parkinson's disease and Alzheimer's disease.

### SUMMARY

A method and system for diagnosing Parkinson's disease and Alzheimer's disease is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a diagnostic kit includes a high flow nitrocellulose membrane configured for lateral flow in a first direction via capillary action. A sample loading area is located at a first end of the membrane and upstream of the first direction. The sample loading area is configured to absorb blood serum or blood plasma from a patient. A set of anti-CD38 antibodies conjugated to colloidal gold nanoparticles are located at a first location on the membrane and downstream of the sample loading area. The first location is configured to change color when the blood serum or blood plasma from the patient contains CD38 at a concentration of at least 1 ng/ml. A set of unconjugated anti-CD38 antibodies are located at a second location on the membrane and downstream of the first location. The second location is configured to change color when the blood serum or blood plasma from the patient contains CD38 at a concentration of at least 1 ng/ml. A cellulose absorbent cloth is located at a second end of the membrane and downstream of the first direction to aid the lateral flow. When the sample loading area has absorbed the blood serum or blood plasma from the patient, the blood serum or blood plasma travels in the first direction along the high flow nitrocellulose membrane towards the first location and the second location on the membrane.

In another embodiment, a diagnostic kit includes a high flow nitrocellulose membrane configured for lateral flow in a first direction via capillary action. A sample loading area is located at a first end of the membrane and upstream of the first direction. The sample loading area is configured to absorb blood serum or blood plasma from a patient. A set of anti-CD38 antibodies conjugated to colloidal gold nanoparticles are located at a first location on the membrane and downstream of the sample loading area. The first location is configured to change color when the blood serum or blood plasma from the patient contains CD38 at a concentration of at least 1 ng/ml. A set of anti-alpha-synuclein antibodies conjugated to colloidal gold nanoparticles are located at the first location on the membrane. The first location is configured to change color when the blood serum or blood plasma from the patient contains alpha-synuclein at a concentration of at least 15 ng/ml. A set of unconjugated anti-CD38 antibodies are located at a second location on the membrane and downstream of the first location. The second location is configured to change color when the blood serum or blood plasma from the patient contains CD38 at a concentration of at least 1 ng/ml. A set of unconjugated anti-alpha-synuclein antibodies are located at a third location on the membrane and downstream of the first and second locations. The third location is configured to change color when the blood serum or blood plasma from the patient contains alpha-synuclein at a concentration of at least 15 ng/ml. A cellulose absorbent cloth is located at a second end of the membrane and downstream of the first direction to aid the lateral flow. When the sample loading area has absorbed the blood serum or blood plasma from the patient, the blood serum or blood plasma travels in the first direction along the high flow nitrocellulose membrane towards the first location, second location and third location on the membrane.

In yet another embodiment, a diagnostic kit includes a high flow nitrocellulose membrane configured for lateral flow in a first direction via capillary action. A sample loading area is located at a first end of the membrane and upstream of the first direction. The sample loading area is configured to absorb blood serum or blood plasma from a patient. A set of anti-CD38 antibodies conjugated to colloidal gold nanoparticles are located at a first location on the membrane and downstream of the sample loading area. The first location is configured to change color when the blood serum or blood plasma from the patient contains CD38 at a concentration of at least 1 ng/ml. A set of anti-tau antibodies conjugated to colloidal gold nanoparticles are located at the first location on the membrane. The first location is configured to change color when the blood serum or blood plasma from the patient contains tau at a concentration of at least 62 ng/ml. A set of unconjugated anti-CD38 antibodies are located at a second location on the membrane and downstream of the first location. The second location is configured to change color when the blood serum or blood plasma from the patient contains CD38 at a concentration of at least 1 ng/ml. A set of unconjugated anti-tau antibodies located at a third location on the membrane and downstream of the first and second locations. The third location is configured to change color when the blood serum or blood plasma from the patient contains tau at a concentration of at least 62 ng/ml. A cellulose absorbent cloth is located at a second end of the membrane and downstream of the first direction to aid the lateral flow. When the sample loading area has absorbed the blood serum or blood plasma from the patient, the blood serum or blood plasma travels in the first direction along the high flow nitrocellulose membrane towards the first location, second location and third location on the membrane.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 is a flowchart describing the steps of the process for diagnosing Parkinson's disease or Alzheimer's disease, according to an example embodiment of the present invention;

FIG. 2 is a graphical representation of a lateral flow diagnostic for Parkinson's disease or Alzheimer's disease, according to an example embodiment of the present invention;

FIG. 3 is a graphical representation of the lateral flow diagnostic of FIG. 2, wherein a blood sample has been applied to the sample application area, according to an example embodiment of the present invention;

FIG. 4 is a graphical representation of the lateral flow diagnostic of FIG. 2, wherein the blood sample is travelling laterally, according to an example embodiment of the present invention;

FIG. 5 is a graphical representation of the lateral flow diagnostic of FIG. 2, wherein a CD38 biomarker is binding with anti-CD38 antibodies conjugated to colloidal gold nanoparticles resulting in the formation of a first line, according to an example embodiment of the present invention;

FIG. 6 is a graphical representation of the lateral flow diagnostic of FIG. 2, wherein the CD38 biomarker and anti-CD38 antibodies conjugated to colloidal gold nanoparticles are moving laterally, according to an example embodiment of the present invention;

FIG. 7 is a graphical representation of the lateral flow diagnostic of FIG. 2, wherein the CD38 biomarker and anti-CD38 antibodies conjugated to colloidal gold nanoparticles binding with unconjugated anti-CD38 antibodies resulting in the formation of a second line, according to an example embodiment of the present invention;

FIG. 8 is a graphical representation of a lateral flow diagnostic for Parkinson's disease, according to an example embodiment of the present invention;

FIG. 9 is a graphical representation of the lateral flow diagnostic of FIG. 8, wherein a blood a sample (plasma or serum) has been applied to the sample application area, according to an example embodiment of the present invention;

FIG. 10 is a graphical representation of the lateral flow diagnostic of FIG. 8, wherein the blood sample is moving laterally down the nitrocellulose membrane, according to an example embodiment of the present invention;

FIG. 11 is a graphical representation of the lateral flow diagnostic of FIG. 8, wherein Parkinson's disease biomarkers (CD38 and alpha-synuclein) are binding with biomarker specific antibodies (anti-alpha-synuclein and anti-CD38) conjugated to colloidal gold nanoparticles resulting in the formation of a first line, according to an example embodiment of the present invention;

FIG. 12 is a graphical representation of the lateral flow diagnostic of FIG. 8, wherein Parkinson's disease biomarkers (CD38 and alpha-synuclein) and biomarker specific antibodies (anti-CD38 and anti-alpha-synuclein) conjugated to colloidal gold nanoparticles moving laterally down the nitrocellulose membrane, according to an example embodiment of the present invention;

FIG. 13 is a graphical representation of the lateral flow diagnostic of FIG. 8, wherein Parkinson's disease biomarkers (CD38 and alpha-synuclein) and biomarker specific antibodies (anti-CD38 and anti-alpha-synuclein) conjugated to colloidal gold nanoparticles binding with unconjugated biomarker specific antibodies (anti-CD38 and anti-alpha-synuclein) resulting in the formation of two lines, according to an example embodiment of the present invention;

FIG. 14 is a graphical representation of a lateral flow diagnostic for Alzheimer's disease, according to an example embodiment of the present invention;

FIG. 15 is a graphical representation of the lateral flow diagnostic of FIG. 14, wherein a blood sample (plasma or serum) has been applied to the sample application area, according to an example embodiment of the present invention;

FIG. 16 is a graphical representation of the lateral flow diagnostic of FIG. 14, wherein a sample (plasma or serum) is moving laterally down the nitrocellulose membrane, according to an example embodiment of the present invention;

FIG. 17 is a graphical representation of the lateral flow diagnostic of FIG. 14, wherein Alzheimer's disease biomarkers (CD38 and tau) binding with biomarker specific antibodies (anti-CD38 and anti-tau) conjugated to colloidal gold nanoparticles resulting in the formation of a first line, according to an example embodiment of the present invention;

FIG. 18 is a graphical representation of the lateral flow diagnostic of FIG. 14, wherein Alzheimer's disease biomarkers (CD38 and alpha-synuclein) and biomarker specific antibodies (anti-CD38 and anti-tau) conjugated to colloidal gold nanoparticles moving laterally down the nitrocellulose membrane, according to an example embodiment of the present invention; and

FIG. 19 is a graphical representation of the lateral flow diagnostic of FIG. 14, wherein Alzheimer's disease biomarkers (CD38 and alpha-synuclein) and biomarker specific antibodies (anti-CD38 and anti-tau) conjugated to colloidal gold nanoparticles binding with unconjugated biomarker specific antibodies (anti-CD38 and anti-tau) resulting in the formation of two lines, according to an example embodiment of the present invention.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The present invention meets the need for a rapid cost-effective diagnostic indicator for Parkinson's disease and Alzheimer's disease. The diagnostic is based on the findings that the CD38 protein is found in significantly higher concentrations in individuals with Parkinson's disease and Alzheimer's disease as compared to non-Parkinson's disease and non-Alzheimer's disease individuals. Since CD38 is significantly elevated in both Parkinson's disease and Alzheimer's individuals it will serve as the biomarker and the basis of the diagnostic. Elevated CD38 protein levels was observed in Western Blot Analysis using whole brain lysates in the serum.

To initially assay for alpha-synuclein in serum samples, serum was collected from individuals that were not affected by Parkinson's or Alzheimer's disease, individuals that were affected by Parkinson's disease and individuals that were affected by Alzheimer's disease. The serum was subjected to Western Blot analysis looking for the alpha-synuclein protein. These data demonstrate that plasma alpha-synuclein is only detectable in Parkinson's disease individuals, not in non-disease state individuals or in Alzheimer's disease individuals. With this information, alpha-synuclein can be used as a second biomarker in conjunction with CD38 to specifically diagnose Parkinson's disease.

To initially explore the feasibility of utilizing the tau proteins as a potential confirmatory/ancillary diagnostic biomarker for Alzheimer's disease to be used in conjunction with CD38, Western Blot Analysis was performed on serum samples from non-disease state individuals, Parkinson's disease-afflicted individuals and Alzheimer's disease-afflicted individuals looking for the presence of tau in the serum. These data demonstrate that the tau protein is only present in the Alzheimer's disease-afflicted individual.

Additionally, to further establish the feasibility of utilizing the tau protein as a potential biomarker to confirm the Alzheimer's diagnostic, plasma samples were assayed by lateral flow, looking at the tau protein specifically, to determine if there was a concentration difference between non-Alzheimer' s and Alzheimer's disease individuals and Parkinson's disease individuals so as to establish an Alzheimer's specific confirmatory biomarker to use in conjunction with CD38. As demonstrated below, the tau protein was only detectable at the pathological concentration in the plasma samples from the Alzheimer's disease-afflicted individual. These data demonstrate that the tau protein can be used as an Alzheimer's disease specific biomarker as a confirmatory test in conjunction with CD38.

Since CD38 is ubiquitously expressed, the specific concentration of the CD38 protein in normal non-disease state individuals must be quantitated and compared to that of both Parkinson's disease individuals and Alzheimer's disease individuals in order to establish a CD38 pathogenic concentration range. This concentration range will be used to diagnose either disease state. To this end, Enzyme Linked Immunosorbent Assays were performed to quantitate the amount of CD38 protein in Parkinson's disease serum, Alzheimer's disease serum and non-disease state individuals.

Additionally, in one study, serum samples (50 age-matched normal controls, 50 Alzheimer's disease patients and 50 Parkinson's disease patients) were assayed for CD38, tau, alpha-synuclein and beta-amyloid protein concentrations in serum. The results of the specific concentration ranges of each biomarker can be seen below.

| Disease Biomarker | Normal | | Parkinson's | | Alzheimer's | |
|---|---|---|---|---|---|---|
| Tau | 1-18 | ng/ml | 55-97 | ng/ml | 172-496 | ng/ml |
| Beta-Amyloid | 0-31 | ng/ml | 0-26.89 | ng/ml | 84-300 | ng/ml |
| Alpha-synuclein | 0-1.51 | ng/ml | 7.42-12.9 | ng/ml | 0-2.72 | ng/ml |
| CD38 | 0-14.22 | ng/ml | 139-343 | ng/ml | 312-838 | ng/ml |

The table above shows protein concentration ranges of each biomarker with respect to normal individual's serum samples, Parkinson's disease-afflicted individual's serum and Alzheimer's disease-afflicted individual's serum

The disclosed embodiments are a multiplex diagnostic. One biomarker, CD38, is common to both Alzheimer's and Parkinson's disease, i.e., it is found in elevated concentrations in both Parkinson's and Alzheimer's when compared to non-disease state individuals. To specifically differentiate between the disease states, additional biomarkers need to be utilized. For Parkinson's disease, elevated alpha-synuclein levels in serum or plasma are indicative of Parkinson's when compared to controls. For Alzheimer's disease, elevated tau protein levels are indicative of Alzheimer's disease when compared to controls. Therefore, for a positive Parkinson's disease diagnosis, CD38 and alpha-synuclein must be detected, and for a positive Alzheimer's disease diagnosis, CD38 and tau must be detected. The diagnostic is designed only to detect the pathological levels of said proteins.

The disclosed embodiments for diagnosing Parkinson's disease, as well Alzheimer's disease, employ a rapid lateral flow capture assay to detect the presence of the CD38 protein in blood samples (specifically plasma or serum). Lateral flow tests, also known as lateral flow immunochromatographic assays, are simple devices intended to detect the presence (or absence) of a target analyte in a sample (matrix) without the need for specialized and costly equipment. Typically, these tests are used for medical diagnostics either for home testing, point of care testing, or laboratory use. A widely spread and well-known application is the home pregnancy test.

The disclosed embodiments utilize specific IgG anti-CD38 antibodies at a specific concentration required to detect a minimum of 1 ng/ml of the CD38 protein conjugated to colloidal gold nanoparticles attached directly to a high flow nitrocellulose membrane (i.e., lateral flow test) at a sample loading area where the nitrocellulose membrane binds proteins electrostatically through interactions of the strong dipole of the nitrate ester with the strong dipole of the peptide bond of the protein, wherein the interaction is completely independent of the pH.

It is important to note that the CD38 protein concentration of at least 1 ng/ml is not recognized as a particular concentration range that can be used as a diagnostic indicator for Parkinson's disease and Alzheimer's disease. As discussed above, the specific concentration of the CD38 protein in normal non-disease state individuals was quantitated and compared to that of both Parkinson's disease individuals and Alzheimer's disease individuals. The CD38 protein concentration of at least 1 ng/ml is significant because it is the result of over 18 months of laboratory and experimental testing of the claimed invention to establish a CD38 pathogenic concentration range for diagnosing either disease state. Thus, if the CD38 protein concentration is less than the threshold concentration indicative of the disease states (<1ng/ml), it will yield a negative result. Further, the concentration of CD38 in a patient sample is not recognized in the prior art as a result effective variable in detecting abnormalities. In other words, it is not well known by persons of ordinary skill in the field of medical diagnostic technology that the concentration of CD38 in a patient sample is a variable that can be varied to obtain desired results in the nature of diagnostics.

The disclosed embodiments also include antibodies that are affinity-purified and a nitrocellulose membrane with a specific pore size and capillary flow rate. The nitrocellulose membrane also includes a specific overall total length. Moreover, a second anti-CD38 antibody is located downstream from the first anti-CD38 antibody that functions as a capture line. Additionally, the disclosed embodiments employ a bed for a cellulose filter paper, binding buffers devoid of Tween 20 and Triton X-100 due the inhibitory binding activity of these compounds, and a specific membrane whose lateral flow rate is specific as a function of antibody-antigen complex formation, where: R=k[AB][Ag] (where R=flow rate, k=constant, AB=antibody, and AG=antigen). Significantly, doubling the flow rate leads to a four-fold decrease in the concentration of the complex, which would make detection of the complex formation more difficult: R=k[0.5×Ab][0.5×Ag]=0.25[Ab][Ag].

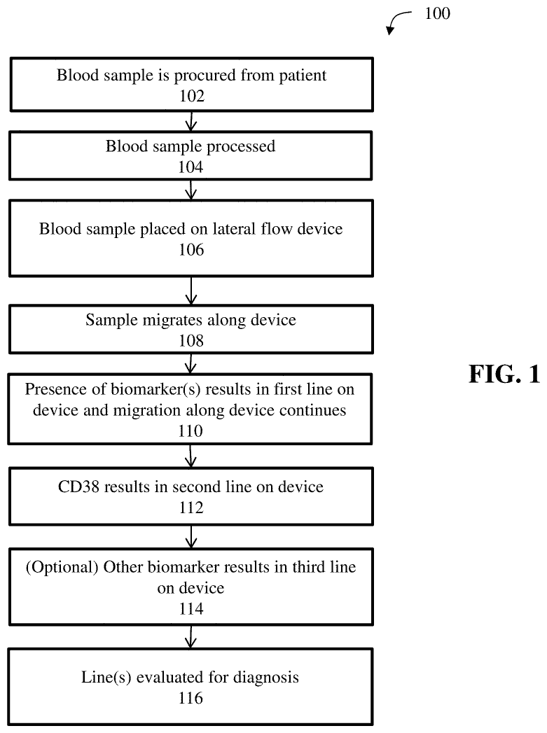

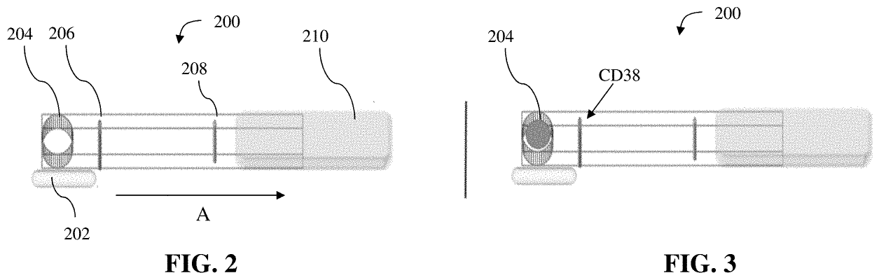

Referring now to the Figures, FIG. 1 is a flowchart describing the steps of the process 100 for diagnosing a Parkinson's or Alzheimer's disease, according to an example embodiment of the present invention. FIGS. 2-7 are illustrations of a nitrocellulose membrane in various stages of the process for diagnosing a Parkinson's or Alzheimer's disease, according to an example embodiment of the present invention. The diagnostic kit in FIGS. 2-7 is based on the findings that the CD38 protein is found in significantly higher concentrations in individuals with Parkinson's disease and Alzheimer's disease as compared to non-Parkinson's disease and non-Alzheimer's disease individuals. Thus, since the CD38 protein is significantly elevated in both Parkinson's disease and Alzheimer's individuals, the CD38 protein serves as the biomarker and basis of the diagnostic in FIGS. 2-7.

In step 102, the process begins by procuring a blood sample from the suspected Parkinson's or Alzheimer's disease-afflicted individual. Next, in step 104, the blood sample is processed to acquire either serum or plasma. If the individual has Parkinson's or Alzheimer's disease, they will have a substantial increase in CD38 protein levels in this source material. In step 106, the serum or plasma is applied to a sample application area 204 on a lateral flow device 200. As shown in FIGS. 2 and 3, the diagnostic kit includes a high flow nitrocellulose membrane configured for lateral flow in a first direction via capillary action. The sample loading area is located at a first end of the membrane and upstream of a first direction. The sample loading area is configured to absorb blood serum or blood plasma from a patient.

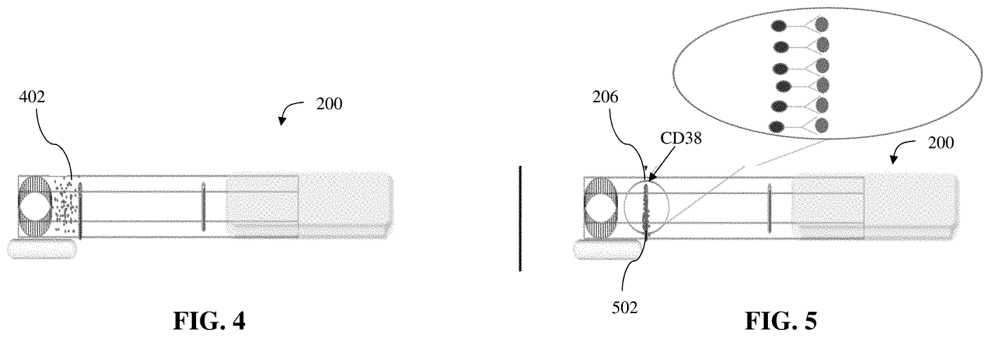

In step 108, when the sample loading area has absorbed the blood serum or blood plasma from the patient, the blood serum or blood plasma 402 travels in a first direction (arrowed line A) along the high flow nitrocellulose membrane toward a first location 206, as shown in FIG. 4. A set of anti-CD38 antibodies conjugated to colloidal gold nanoparticles are located at the first location on the membrane and downstream of the sample loading area. The first location is configured to change color when the blood serum or blood plasma from the patient contains CD38 at a concentration of at least 1 ng/ml.

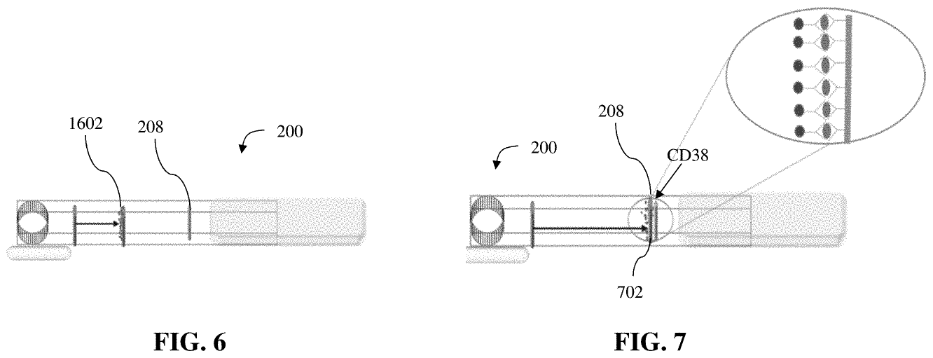

In step 110, if the pathological concentration contains CD38 at a concentration of at least 1 ng/ml, it will bind irreversibly to the anti-CD38 antibodies conjugated to the colloidal gold nanoparticles and form a CD38-antibody-colloidal gold complex 1602 resulting in a first line 502 and continue migrating with the protein down the membrane, as shown in FIGS. 5 and 6. The CD38 protein-CD38 antibody conjugated to the colloidal gold 1602 will migrate until it interacts with a second location 208. A set of unconjugated anti-CD38 antibodies are located at a second location on the membrane and downstream of the first location. The second location is configured to change color when the blood serum or blood plasma from the patient contains CD38 at a concentration of at least 1 ng/ml.

In step 112, if the blood serum or blood plasma from the patient contains CD38 at a concentration of at least 1 ng/ml, it will be captured by the unconjugated antibody and the result will be the presence of a second line 702 on the nitrocellulose membrane, as shown in FIG. 7. If CD38 is at a concentration of less than 1 ng/ml, there will not be the formation of a second line, thereby yielding a negative result on the either the Parkinson's or Alzheimer's disease diagnostic. In step 116, the lines are evaluated for diagnosis. The necessary concentration of CD38 indicative of the disease states will result in two lines, but the insufficient concentrations of CD38 indicative of the non-disease states will not result in two lines. Consequently, two lines in the Parkinson's or Alzheimer's disease diagnostic membrane results in a partial diagnosis of a Parkinson's or Alzheimer's disease.

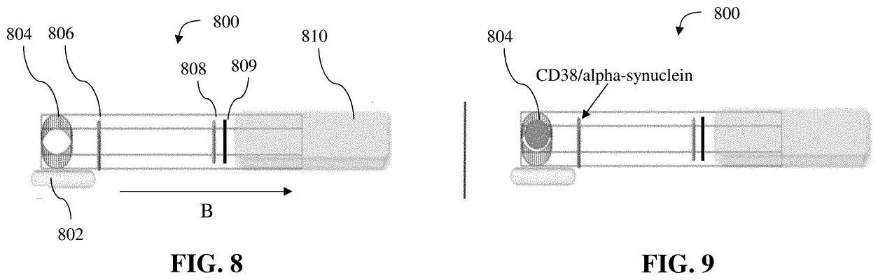

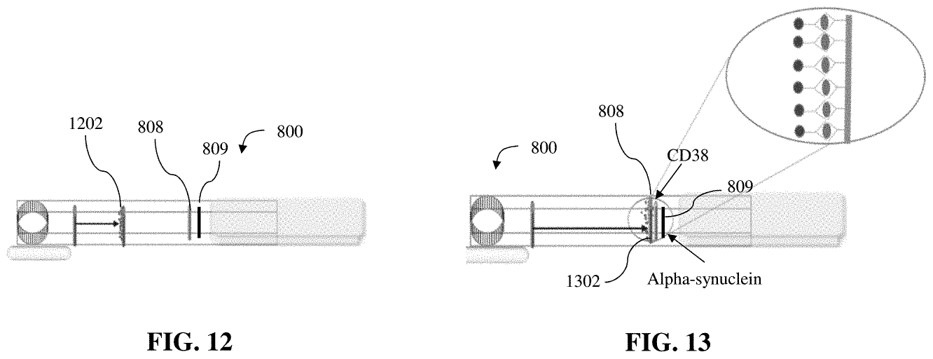

FIGS. 8-13 are illustrations of a nitrocellulose membrane in various stages of the process for diagnosing Parkinson's disease, to an example embodiment of the present invention. The diagnostic kit in FIGS. 8-13 is based on the findings that the CD38 protein is found in significantly higher concentrations in individuals with Parkinson's disease and Alzheimer's disease as compared to non-Parkinson's disease and non-Alzheimer's disease individuals. However, to specifically differentiate between the disease states, additional biomarkers need to be utilized. For Parkinson's disease, elevated alpha-synuclein levels in serum or plasma are indicative of Parkinson's when compared to controls. Therefore, CD38 and alpha-synuclein serve as the biomarkers and basis of the diagnostic in FIGS. 8-13.

It is important to note that the alpha-synuclein concentration of at least 15 ng/ml is not recognized as a particular concentration range that can be used as a diagnostic indicator for Parkinson's disease. As discussed above, the specific concentration of the alpha-synclein protein in normal non-disease state individuals was quantitated and compared to that of both Parkinson's disease individuals. The alpha-synuclein protein concentration of at least 15 ng/ml is significant because it is the result of over 18 months of laboratory and experimental testing of the claimed invention to establish an alpha-synuclein pathogenic concentration range for diagnosing either disease state. Thus, if the alpha-synuclein protein concentration is less than the threshold concentration indicative of the disease states (<15ng/ml), it will yield a negative result. Further, the concentration of alpha-synuclein in a patient sample is not recognized in the prior art as a result effective variable in detecting abnormalities. In other words, it is not well known by persons of ordinary skill in the field of medical diagnostic technology that the concentration of alpha-synuclein in a patient sample is a variable that can be varied to obtain desired results in the nature of diagnostics.

In step 102, the process begins by procuring a blood sample from a suspected Parkinson's disease-afflicted individual. Next, in step 104, the blood sample is processed to acquire either serum or plasma. If the individual has Parkinson's disease, they will have a substantial increase in CD38 protein levels and alpha-synuclein levels in this source material. In step 106, the serum or plasma is applied to a sample application area 804 on a lateral flow device 800. As shown in FIGS. 8 and 9, the diagnostic kit includes a high flow nitrocellulose membrane configured for lateral flow in a first direction via capillary action. A sample loading area is located at a first end of the membrane and upstream of a first direction. The sample loading area is configured to absorb blood serum or blood plasma from a patient.

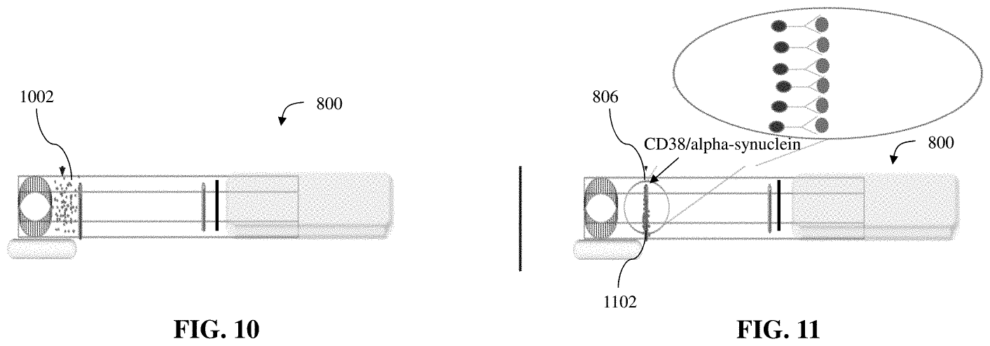

In step 108, when the sample loading area has absorbed the blood serum or blood plasma from the patient, the blood serum or blood plasma 1002 travels in a first direction (arrowed line B) along the high flow nitrocellulose membrane toward a first location 806, as shown in FIG. 10. A set of anti-CD38 antibodies conjugated to colloidal gold nanoparticles are located at the first location on the membrane and downstream of the sample loading area. The first location is configured to change color when the blood serum or blood plasma from the patient contains CD38 at a concentration of at least 1 ng/ml. Additionally, a set of anti-alpha-synuclein antibodies conjugated to colloidal gold nanoparticles are located at the first location on the membrane. The first location is configured to change color when the blood serum or blood plasma from the patient contains alpha-synuclein at a concentration of at least 15 ng/ml.

In step 110, if the pathological concentration contains CD38 at a concentration of at least 1 ng/ml, it will bind irreversibly to the anti-CD38 antibodies conjugated to the colloidal gold nanoparticles and form a CD38-antibody-colloidal gold complex 1102 resulting in a first line and continue migrating 1202 with the protein down the membrane until it interacts with a second location 808, as shown in FIGS. 5 and 6. Likewise, if the pathological concentration contains alpha-synuclein at a concentration of at least 15 ng/ml, it will bind irreversibly to the anti-alpha-synuclein antibodies conjugated to the colloidal gold nanoparticles and form an alpha-synuclein-antibody-colloidal gold complex and continue migrating with the protein down the membrane until it interacts with a third location 809, as shown in FIGS. 5 and 6.

A set of unconjugated anti-CD38 antibodies are located at a second location on the membrane and downstream of the first location. The second location is configured to change color when the blood serum or blood plasma from the patient contains CD38 at a concentration of at least 1 ng/ml. A set of unconjugated anti-CD38 antibodies are located at a second location on the membrane and downstream of the first location. The second location is configured to change color when the blood serum or blood plasma from the patient contains CD38 at a concentration of at least 1 ng/ml. Additionally, a set of unconjugated anti-alpha-synuclein antibodies are located at a third location on the membrane and downstream of the first and second locations. The third location is configured to change color when the blood serum or blood plasma from the patient contains alpha-synuclein at a concentration of at least 15 ng/ml.

In step 112, if the necessary concentration of CD38 protein is present, which will be the one of the indicators of this diagnostic, it will be captured by the unconjugated antibody and the result will be the presence of a second line on the nitrocellulose membrane. Additionally, in step 114, if the necessary concentration of the alpha-synuclein protein is present, which will be the other indicator of the Parkinson's disease diagnostic, it will be captured by the unconjugated alpha-synuclein antibody and the result will be the presence of a third line 1302 on the nitrocellulose membrane.

In step 116, if CD38 protein concentration is less than the threshold concentration indicative of the disease states (<1 ng/ml), there will not be the formation of a second line thereby yielding a negative result on Parkinson's disease diagnostic. Additionally, if the alpha-synuclein protein concentration is less than the threshold concentration indicative of the disease state (<15 ng/ml), there will not be the formation of the third line, thereby yielding a negative result on the Parkinson's diagnostic. Thus, the necessary protein concentrations of CD38 and alpha-synuclein indicative of the disease state will result in three lines (control, CD38 and alpha-synuclein), but the insufficient protein concentrations of CD38 and alpha-synuclein indicative of the non-disease states will not result in two lines, rather just one line representing the lateral flow control line. Consequently, three lines in the Parkinson's disease diagnostic membrane results in a diagnosis of a Parkinson's disease whereas the formation of only one line indicates a negative test result for Parkinson's disease.

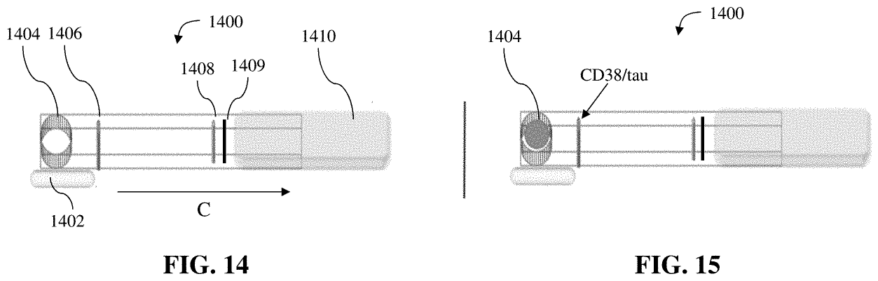

FIGS. 14-19 are illustrations of a nitrocellulose membrane in various stages of the process for diagnosing Alzheimer's disease, according to one embodiment. FIGS. 14-19 are illustrations of a nitrocellulose membrane in various stages of the process for diagnosing Alzheimer's disease, to an example embodiment of the present invention. The diagnostic kit in FIGS. 14-19 is based on the findings that the CD38 protein is found in significantly higher concentrations in individuals with Parkinson's disease and Alzheimer's disease as compared to non-Parkinson's disease and non-Alzheimer's disease individuals. However, to specifically differentiate between the disease states, additional biomarkers need to be utilized. For Alzheimer's disease, elevated tau levels in serum or plasma are indicative of Alzheimer's when compared to controls. Therefore, CD38 and tau serve as the biomarkers and basis of the diagnostic in FIGS. 14-19.

It is important to note that the tau concentration of at least 62 ng/ml is not recognized as a particular concentration range that can be used as a diagnostic indicator for Alzheimer's disease. As discussed above, the specific concentration of the tau protein in normal non-disease state individuals was quantitated and compared to that of both Alzheimer's disease individuals. The tau protein concentration of at least 62 ng/ml is significant because it is the result of over 18 months of laboratory and experimental testing of the claimed invention to establish a tau pathogenic concentration range for diagnosing either disease state. Thus, if the tau protein concentration is less than the threshold concentration indicative of the disease states (<62 ng/ml), it will yield a negative result. Further, the concentration of tau in a patient sample is not recognized in the prior art as a result effective variable in detecting abnormalities. In other words, it is not well known by persons of ordinary skill in the field of medical diagnostic technology that the concentration of tau in a patient sample is a variable that can be varied to obtain desired results in the nature of diagnostics.

In step 102, the process begins by procuring a blood sample from a suspected Alzheimer's disease-afflicted individual. Next, in step 104, the blood sample is processed to acquire either serum or plasma. If the individual has Alzheimer's disease, they will have a substantial increase in CD38 protein levels and tau levels in this source material. In step 106, the serum or plasma is applied to a sample application area 1404 on a lateral flow device 1400. As shown in FIGS. 8 and 9, the diagnostic kit includes a high flow nitrocellulose membrane configured for lateral flow in a first direction via capillary action. A sample loading area is located at a first end of the membrane and upstream of the first direction. The sample loading area is configured to absorb blood serum or blood plasma from a patient.

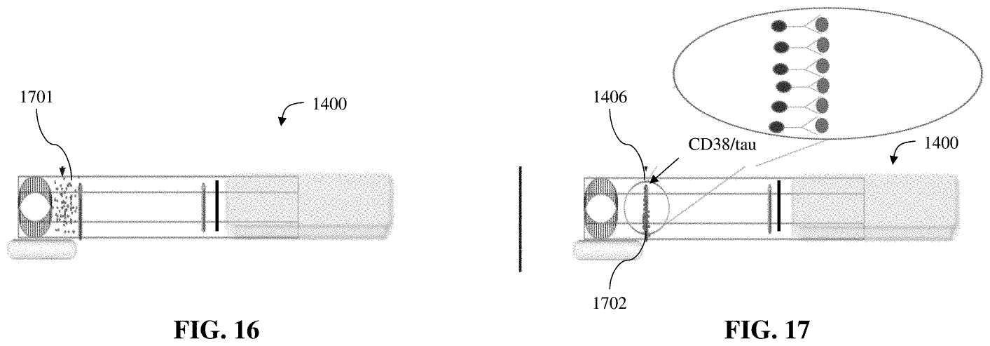

In step 108, when the sample loading area has absorbed the blood serum or blood plasma from the patient, the blood serum or blood plasma 1701 travels in a first direction (arrowed line C) along the high flow nitrocellulose membrane toward a first location 1406, as shown in FIG. 16. A set of anti-CD38 antibodies conjugated to colloidal gold nanoparticles are located at the first location on the membrane and downstream of the sample loading area. The first location is configured to change color when the blood serum or blood plasma from the patient contains CD38 at a concentration of at least 1 ng/ml. Additionally, a set of anti-tau antibodies conjugated to colloidal gold nanoparticles are located at the first location on the membrane. The first location is configured to change color when the blood serum or blood plasma from the patient contains tau at a concentration of at least 62 ng/ml.

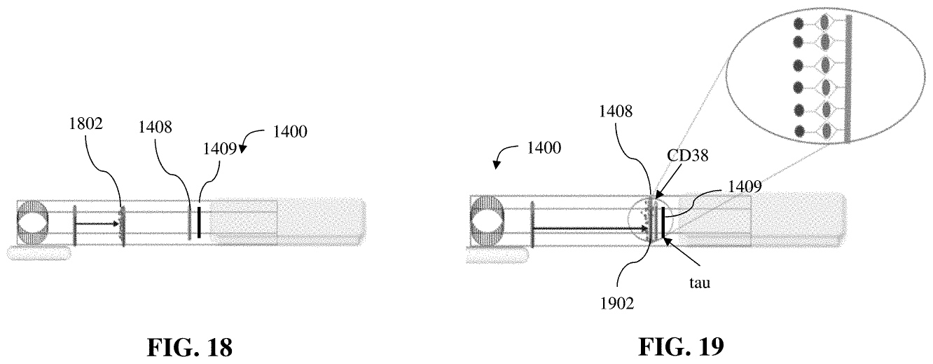

In step 110, if the pathological concentration contains CD38 at a concentration of at least 1 ng/ml, it will bind irreversibly to the anti-CD38 antibodies conjugated to the colloidal gold nanoparticles and form a CD38-antibody-colloidal gold complex 1702 resulting in a first line and continue migrating 1802 with the protein down the membrane until it interacts with a second location 1408, as shown in FIGS. 17 and 18. Likewise, if the pathological concentration contains tau at a concentration of at least 62 ng/ml, it will bind irreversibly to the anti-tau antibodies conjugated to the colloidal gold nanoparticles and form a tau-antibody-colloidal gold complex and continue migrating with the protein down the membrane until it interacts with a third location 1409, as shown in FIGS. 17 and 18.

A set of unconjugated anti-CD38 antibodies are located at a second location on the membrane and downstream of the first location. The second location is configured to change color when the blood serum or blood plasma from the patient contains CD38 at a concentration of at least 1 ng/ml. A set of unconjugated anti-CD38 antibodies are located at a second location on the membrane and downstream of the first location. The second location is configured to change color when the blood serum or blood plasma from the patient contains CD38 at a concentration of at least 1 ng/ml. Additionally, a set of unconjugated anti-tau antibodies are located at a third location on the membrane and downstream of the first and second locations. The third location is configured to change color when the blood serum or blood plasma from the patient contains tau at a concentration of at least 62 ng/ml.

In step 112, if the necessary concentration of CD38 protein is present, which will be the one of the indicators of this diagnostic, it will be captured by the unconjugated antibody and the result will be the presence of a second line on the nitrocellulose membrane. Additionally, in step 114, if the necessary concentration of the tau protein is present, which will be the other indicator of the Alzheimer's disease diagnostic, it will be captured by the unconjugated tau antibody and the result will be the presence of a third line 1902 on the nitrocellulose membrane.

In step 116, if CD38 protein concentration is less than the threshold concentration indicative of the disease states (<1 ng/ml), there will not be the formation of a second line thereby yielding a negative result on Alzheimer's disease diagnostic. Additionally, if the tau protein concentration is less than the threshold concentration indicative of the disease state (<62 ng/ml), there will not be the formation of the third line thereby yielding a negative result on the Alzheimer's diagnostic. Thus, the necessary protein concentrations of CD38 and tau indicative of the disease state will result in three lines (including a lateral flow control line), but the insufficient protein concentrations of CD38 and tau indicative of the non-disease states will not result in two lines. Consequently, three lines in the Alzheimer's disease diagnostic membrane results in a diagnosis of an Alzheimer's disease and one red line (lateral flow control line) is indicative of a negative test result on the Alzheimer's disease diagnostic.

In an alternative embodiment, a nitrocellulose membrane may be use for diagnosing Alzheimer's disease, wherein CD38, beta amyloid and tau serve as the biomarkers and basis of the diagnostic.

In step 102 of this alternative embodiment, the process begins by procuring a blood sample from a suspected Alzheimer's disease-afflicted individual. Next, in step 104, the blood sample is processed to acquire either serum or plasma. If the individual has Alzheimer's disease, they will have a substantial increase in CD38 protein levels, beta amyloid and tau levels in this source material. In step 106, the serum or plasma is applied to a sample application area on a lateral flow device. The diagnostic kit includes a high flow nitrocellulose membrane configured for lateral flow in a first direction via capillary action. A sample loading area is located at a first end of the membrane and upstream of the first direction. The sample loading area is configured to absorb blood serum or blood plasma from a patient.

In step 108, when the sample loading area has absorbed the blood serum or blood plasma from the patient, the blood serum or blood plasma travels in a first direction along the high flow nitrocellulose membrane toward a first location. A set of anti-CD38 antibodies conjugated to colloidal gold nanoparticles are located at the first location on the membrane and downstream of the sample loading area. The first location is configured to change color when the blood serum or blood plasma from the patient contains CD38 at a concentration of at least 1 ng/ml. Additionally, a set of anti-tau antibodies conjugated to colloidal gold nanoparticles are located at the first location on the membrane. Additionally, a set of anti-amyloid beta antibodies conjugated to colloidal gold nanoparticles are located at the first location on the membrane. The first location is configured to change color when the blood serum or blood plasma from the patient contains amyloid beta at a concentration of at least 100 ng/ml.

In step 110, if the pathological concentration contains CD38 at a concentration of at least 1 ng/ml, it will bind irreversibly to the anti-CD38 antibodies conjugated to the colloidal gold nanoparticles and form a CD38-antibody-colloidal gold complex resulting in a first line and continue migrating with the protein down the membrane until it interacts with a second location. Likewise, if the pathological concentration contains tau at a concentration of at least 62 ng/ml, it will bind irreversibly to the anti-tau antibodies conjugated to the colloidal gold nanoparticles and form a tau-antibody-colloidal gold complex and continue migrating with the protein down the membrane until it interacts with a third location. Likewise, if the pathological concentration contains amyloid beta at a concentration of at least 100 ng/ml, it will bind irreversibly to the anti-amyloid beta antibodies conjugated to the colloidal gold nanoparticles and form an amyloid beta-antibody-colloidal gold complex and continue migrating with the protein down the membrane until it interacts with a fourth location.

A set of unconjugated anti-CD38 antibodies are located at a second location on the membrane and downstream of the first location. The second location is configured to change color when the blood serum or blood plasma from the patient contains CD38 at a concentration of at least 1 ng/ml. A set of unconjugated anti-CD38 antibodies are located at a second location on the membrane and downstream of the first location. The second location is configured to change color when the blood serum or blood plasma from the patient contains CD38 at a concentration of at least 1 ng/ml. Additionally, a set of unconjugated anti-tau antibodies are located at a third location on the membrane and downstream of the first and second locations. The third location is configured to change color when the blood serum or blood plasma from the patient contains tau at a concentration of at least 62 ng/ml. Additionally, a set of unconjugated anti-amyloid beta antibodies are located at a fourth location on the membrane and downstream of the first and second locations. The fourth location is configured to change color when the blood serum or blood plasma from the patient contains amyloid beta at a concentration of at least 100 ng/ml.

In step 112, if the necessary concentration of CD38 protein is present, which will be the one of the indicators of this diagnostic, it will be captured by the unconjugated antibody and the result will be the presence of a second line on the nitrocellulose membrane. Additionally, in step 114, if the necessary concentration of the tau protein is present, which will be the other indicator of the Alzheimer's disease diagnostic, it will be captured by the unconjugated tau antibody and the result will be the presence of a third line on the nitrocellulose membrane. Additionally, in step 114, if the necessary concentration of the amyloid beta protein is present, which will be the other indicator of the Alzheimer's disease diagnostic, it will be captured by the unconjugated amyloid beta antibody and the result will be the presence of a fourth line on the nitrocellulose membrane.

In step 116, if CD38 protein concentration is less than the threshold concentration indicative of the disease states (<1 ng/ml), there will not be the formation of a second line thereby yielding a negative result on Alzheimer's disease diagnostic. Additionally, if the tau protein concentration is less than the threshold concentration indicative of the disease state (<62 ng/ml), there will not be the formation of the third line thereby yielding a negative result on the Alzheimer's diagnostic. Additionally, if the amyloid beta protein concentration is less than the threshold concentration indicative of the disease state (<100 ng/ml), there will not be the formation of the fourth line thereby yielding a negative result on the Alzheimer's diagnostic. Thus, the necessary protein concentrations of CD38, amyloid beta and tau indicative of the disease state will result in four lines (including a lateral flow control line), but the insufficient protein concentrations of CD38, amyloid beta and tau indicative of the non-disease states will not result in four lines. Consequently, four lines in the Alzheimer's disease diagnostic membrane results in a diagnosis of an Alzheimer's disease and one red line (lateral flow control line) is indicative of a negative test result on the Alzheimer's disease diagnostic.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A diagnostic kit comprising:
a high flow nitrocellulose membrane configured for lateral flow in a first direction via capillary action;
a sample loading area located at a first end of the membrane and upstream of the first direction, wherein the sample loading area is configured to absorb blood serum or blood plasma from a patient;
a set of anti-CD38 antibodies conjugated to colloidal gold nanoparticles located at a first location on the membrane and downstream of the sample loading area, wherein the first location is configured to change color when the blood serum or blood plasma from the patient contains CD38 at a concentration of at least 1 ng/ml;
a set of unconjugated anti-CD38 antibodies located at a second location on the membrane and downstream of the first location, wherein the second location is configured to change color when the blood serum or blood plasma from the patient contains CD38 at a concentration of at least 1 ng/ml; and
a cellulose absorbent cloth located at a second end of the membrane and downstream of the first direction, so as to aid the lateral flow;
wherein when the sample loading area has absorbed the blood serum or blood plasma from the patient, the blood serum or blood plasma travels in the first direction along the high flow nitrocellulose membrane towards the first location and the second location on the membrane.

2. The diagnostic kit of claim 2, wherein if the first location and second location change color, then a partial diagnosis of Alzheimer's disease and/or Parkinson's disease is indicated.

3. A diagnostic kit comprising:
a high flow nitrocellulose membrane configured for lateral flow in a first direction via capillary action;
a sample loading area located at a first end of the membrane and upstream of the first direction, wherein the sample loading area is configured to absorb blood serum or blood plasma from a patient;
a set of anti-CD38 antibodies conjugated to colloidal gold nanoparticles located at a first location on the membrane and downstream of the sample loading area, wherein the first location is configured to change color when the blood serum or blood plasma from the patient contains CD38 at a concentration of at least 1 ng/ml;
a set of anti-alpha-synuclein antibodies conjugated to colloidal gold nanoparticles located at the first location on the membrane, wherein the first location is configured to change color when the blood serum or blood plasma from the patient contains alpha-synuclein at a concentration of at least 15 ng/ml;
a set of unconjugated anti-CD38 antibodies located at a second location on the membrane and downstream of the first location, wherein the second location is configured to change color when the blood serum or blood plasma from the patient contains CD38 at a concentration of at least 1 ng/ml;
a set of unconjugated anti-alpha-synuclein antibodies located at a third location on the membrane and downstream of the first and second locations, wherein the third location is configured to change color when the blood serum or blood plasma from the patient contains alpha-synuclein at a concentration of at least 15 ng/ml; and
a cellulose absorbent cloth located at a second end of the membrane and downstream of the first direction, so as to aid the lateral flow;
wherein when the sample loading area has absorbed the blood serum or blood plasma from the patient, the blood serum or blood plasma travels in the first direction along the high flow nitrocellulose membrane towards the first location, second location and third location on the membrane.

4. The diagnostic kit of claim 3, wherein if the first location, second location and third location change color, then a diagnosis of Parkinson's disease is indicated.

5. A diagnostic kit comprising:
a high flow nitrocellulose membrane configured for lateral flow in a first direction via capillary action;
a sample loading area located at a first end of the membrane and upstream of the first direction, wherein the sample loading area is configured to absorb blood serum or blood plasma from a patient;
a set of anti-CD38 antibodies conjugated to colloidal gold nanoparticles located at a first location on the membrane and downstream of the sample loading area, wherein the first location is configured to change color when the blood serum or blood plasma from the patient contains CD38 at a concentration of at least 1 ng/ml;
a set of anti-tau antibodies conjugated to colloidal gold nanoparticles located at the first location on the membrane, wherein the first location is configured to change color when the blood serum or blood plasma from the patient contains tau at a concentration of at least 62 ng/ml;
a set of unconjugated anti-CD38 antibodies located at a second location on the membrane and downstream of the first location, wherein the second location is configured to change color when the blood serum or blood plasma from the patient contains CD38 at a concentration of at least 1 ng/ml;
a set of unconjugated anti-tau antibodies located at a third location on the membrane and downstream of the first and second locations, wherein the third location is configured to change color when the blood serum or blood plasma from the patient contains tau at a concentration of at least 62 ng/ml; and
a cellulose absorbent cloth located at a second end of the membrane and downstream of the first direction, so as to aid the lateral flow;
wherein when the sample loading area has absorbed the blood serum or blood plasma from the patient, the blood serum or blood plasma travels in the first direction along the high flow nitrocellulose membrane towards the first location, second location and third location on the membrane.

6. The diagnostic kit of claim 5, wherein if the first location, second location and third location change color, then a diagnosis of Alzheimer's disease is indicated.
